(19) United States
(12) Patent Application Publication
(10) Pub. No.: US 2023/0035775 A1
(43) Pub. Date: Feb. 2, 2023

(54) THERAPEUTIC APPLICATION MANAGEMENT SYSTEM, THERAPEUTIC APPLICATION MANAGEMENT METHOD, THERAPEUTIC APPLICATION MANAGEMENT PROGRAM, AND TERMINAL

(71) Applicant: CureApp, Inc., Tokyo (JP)

(72) Inventor: Yutaka Kohada, Tokyo (JP)

(21) Appl. No.: 17/964,099

(22) Filed: Oct. 12, 2022

Related U.S. Application Data
(63) Continuation of application No. PCT/JP2021/014118, filed on Apr. 1, 2021.

(30) Foreign Application Priority Data
Apr. 13, 2020 (JP) 2020-071923

(51) Int. Cl. G16H 20/00; G16H 10/60

(52) U.S. Cl. CPC G16H 20/00; G16H 10/60

(57) ABSTRACT

A therapeutic application management system includes: an acquisition unit configured to acquire an application ID indicating a therapeutic application which is prescribed for a patient by a medical worker, a patient ID for identifying a patient for whom the therapeutic application is to be prescribed, and use deadline information indicating a deadline to which the therapeutic application is able to be used; a registration unit configured to register the application ID for identifying the therapeutic application, the patient ID, and the use deadline information in a storage unit in correlation with each other; a timekeeping unit configured to count date and time information; and a limitation unit configured to limit use of the therapeutic application which is transmitted to a terminal of the patient when the date and time information counted by the timekeeping unit has passed beyond a date and time determined by the use deadline information.

### TECHNICAL FIELD

This disclosure relates to a therapeutic application management system that manages a therapeutic application, a management method and a management program thereof, and a terminal.

### BACKGROUND

Recently, therapeutic applications for improvement in actions associated with a user's health, treatment of diseases, management of physical conditions, for example, have been developed (for example, Japanese Unexamined Patent Application Publication No. 2001-92876). A therapeutic application is an application that records daily actions of a patient or notifies of a message indicating details of actions which are to be carried out by a patient to promote improvement in disease conditions of the patient based on the actions of the patient. In JP 2001-92876 A, a message to improve actions detrimental to health based on data collected from a person is provided to the person.

Such a therapeutic application is for appropriate treatment based on conditions of a patient. Conditions of a patient can be ascertained better through a doctor's intervention as well as management using a therapeutic application. However, when a therapeutic application is used without a doctor's ascertaining of conditions, there is a likelihood that the therapeutic application will not perform appropriate treatment of a patient.

Therefore, it could be helpful to provide a therapeutic application management system, a therapeutic application management method, and a therapeutic application management program that can reduce a likelihood that a therapeutic application will perform inappropriate treatment of a patient.

### SUMMARY

We thus provide a therapeutic application management system that includes: an acquisition unit configured to acquire an application ID indicating a therapeutic application which is prescribed for a patient by a medical worker, a patient ID for identifying a patient for whom the therapeutic application is to be prescribed, and use deadline information indicating a deadline to which the therapeutic application is able to be used; a registration unit configured to register the application ID for identifying the therapeutic application, the patient ID, and the use deadline information in a storage unit in correlation with each other; a timekeeping unit configured to count date and time information; and a limitation unit configured to limit use of the therapeutic application which is transmitted to a terminal of the patient when the date and time information counted by the timekeeping unit has passed beyond a date and time determined by the use deadline information.

The use deadline information may be a deadline which is determined by the medical worker.

The use deadline information may be information in which the number of days in which the therapeutic application is able to be used after the therapeutic application has been prescribed is defined.

The therapeutic application management system may further include: a reception unit configured to receive request information for requesting an activation code for validating the therapeutic application which is downloaded by the patient using the terminal; an issuing unit configured to issue the activation code for the therapeutic application of the patient in response to the request information; and a code transmitting unit configured to transmit the activation code issued by the issuing unit to the terminal of the patient.

The limitation unit may be configured to limit use of the therapeutic application by transmitting a use limitation code for limiting start of the therapeutic application to the terminal of the patient when the number of days indicated by the use deadline information has elapsed from the date and time at which the therapeutic application has been prescribed based on the date and time information counted by the timekeeping unit.

The therapeutic application management system may further include an application transmitting unit configured to transmit a therapeutic application in response to a request from a terminal of a patient.

The limitation unit may be configured to limit use of the therapeutic application by embedding a user limitation code for disabling start of the therapeutic application in the therapeutic application and transmitting the therapeutic application to the terminal of the patient when the use deadline determined by the use deadline information has elapsed.

The therapeutic application management system may further include a suggestion unit configured to suggest a use deadline of the therapeutic application prescribed for the patient to the medical worker based on treatment details received from the medical worker using a trained model having learned a relationship between treatment details of the therapeutic application and a use deadline to which the therapeutic application is able to be used.

The storage unit may be configured to store the treatment details and a basic number of days until the use deadline to which the therapeutic application is able to be used in association with the treatment details in correlation, and the suggestion unit may be configured to suggest a use deadline obtained by adding a correction number of days estimated using the trained model to the basic number of days corresponding to the treatment details received from the medical worker.

The therapeutic application management system may further include: a change unit configured to change a use deadline; and a change information transmitting unit configured to transmit the changed use deadline and an application ID of the therapeutic application of which the use deadline is changed to the terminal of the patient.

The therapeutic application management system may further include a change unit configured to change a use deadline, and the limitation unit may be configured to limit use of the therapeutic application based on the changed use deadline.

The therapeutic application management system may further include a notification unit configured to notify the terminal of the patient using the therapeutic application in which the number of days obtained by subtracting a date indicated by the date and time information from a deadline indicated by the use deadline is equal to or less than a predetermined number of days of information indicating that the use deadline of the therapeutic application is imminent.

We also provide a terminal that includes: a reception unit configured to receive a therapeutic application including use deadline information indicating a use deadline; a display unit configured to display treatment information indicating treatment details determined by the therapeutic application; and a limitation unit configured to limit use of the therapeutic application when the use deadline indicated by the use deadline information expires.

We further provide a therapeutic application management method that is performed by a therapeutic application management system and includes: an acquisition step of acquiring an application ID indicating a therapeutic application which is prescribed for a patient by a medical worker, a patient ID for identifying a patient for whom the therapeutic application is to be prescribed, and use deadline information indicating a deadline to which the therapeutic application is able to be used; a registration step of registering the application ID for identifying the therapeutic application, the patient ID, and the use deadline information in a storage unit in correlation with each other; a timekeeping step of counting date and time information; and a limitation step of limiting use of the therapeutic application which is transmitted to a terminal of the patient when the date and time information counted in the timekeeping step has passed beyond a date and time determined by the use deadline information.

We still further provide a management method of a therapeutic application for a terminal that includes: a reception step of receiving a therapeutic application including use deadline information indicating a use deadline; a display step of displaying treatment information indicating treatment details determined by the therapeutic application; and a limitation step of limiting use of the therapeutic application when the use deadline indicated by the use deadline information expires.

We yet further provide a management program that causes a computer to perform: an acquisition function of acquiring an application ID indicating a therapeutic application which is prescribed for a patient by a medical worker, a patient ID for identifying a patient for whom the therapeutic application is to be prescribed, and use deadline information indicating a deadline to which the therapeutic application is able to be used; a registration function of registering the application ID for identifying the therapeutic application, the patient ID, and the use deadline information in a storage unit in correlation with each other; a timekeeping function of counting date and time information; and a limitation function of limiting use of the therapeutic application which is transmitted to a terminal of the patient when the date and time information counted by the timekeeping function has passed beyond a date and time determined by the use deadline information.

We still yet further provide a management program that causes a computer to perform: a reception function of receiving a therapeutic application including use deadline information indicating a use deadline; a display function of displaying treatment information indicating treatment details determined by the therapeutic application; and a limitation function of limiting use of the therapeutic application when the use deadline indicated by the use deadline information expires.

Since the therapeutic application management system can manage a therapeutic application based on a use deadline which has been designated in advance, it is possible to prevent a patient from misusing a therapeutic application.

### BRIEF DESCRIPTION OF THE DRAWINGS

FIG. 1 is a system diagram schematically illustrating a therapeutic application management system.

FIG. 2 is a block diagram illustrating an example of a configuration of a management server.

FIG. 3 is a block diagram illustrating an example of a configuration of a user terminal.

FIG. 4 is a block diagram illustrating an example of a configuration of a delivery server.

FIG. 5 is a block diagram illustrating an example of a configuration of a terminal of a medical worker.

FIG. 6 is a block diagram illustrating an example of a configuration of an operation server.

FIG. 7 is a conceptual diagram of data illustrating an example of a data structure of management information.

FIG. 8 is a sequence diagram illustrating an example of communication between devices in the therapeutic application management system.

FIG. 9 is a flowchart illustrating an example of a routine which is performed by the management server.

FIG. 10 is a flowchart illustrating an example of a routine which is performed by the user terminal.

FIG. 11 is a flowchart illustrating an example of a routine which is performed by the delivery server.

FIG. 12 is a flowchart illustrating an example of a routine which is performed by the terminal of a medical worker.

FIG. 13 is a flowchart illustrating an example of a routine which is performed by the operation server.

FIG. 14 is a system diagram illustrating another example of the configuration of the therapeutic application management system.

FIG. 15 is a sequence diagram illustrating an example of communication between devices in the therapeutic application management system with the configuration illustrated in FIG. 12.

FIG. 16A is a screen view illustrating an example of a notification of a use deadline.

FIG. 16B is a screen view illustrating an example of display of a therapeutic application.

FIG. 16C is a screen view illustrating another example of display of a therapeutic application.

### REFERENCE SIGNS LIST

100 Management server
110, 210, 310, 510 Reception unit
120, 220, 320, 520 Input unit
130, 230, 330, 530 Control unit
131 Acquisition unit
132 Registration unit
133 Timekeeping unit
134 Limitation unit
135 Issuing unit
136 Suggestion unit
137 Change unit
138 Notification unit
140, 240, 340, 540 Storage unit
141 Management information
150, 250, 350, 550 Output unit
200 User terminal
231 Application executing unit
232 Application limiting unit
300 Delivery server
331 Delivery unit
341 First therapeutic application installer
342 Second therapeutic application installer
400 Network
500 Medical worker terminal
531 Use deadline information transmitting unit

### DETAILED DESCRIPTION

Hereinafter, an information processing device according to an aspect of the present disclosure will be described in detail with reference to the accompanying drawings.

### Example

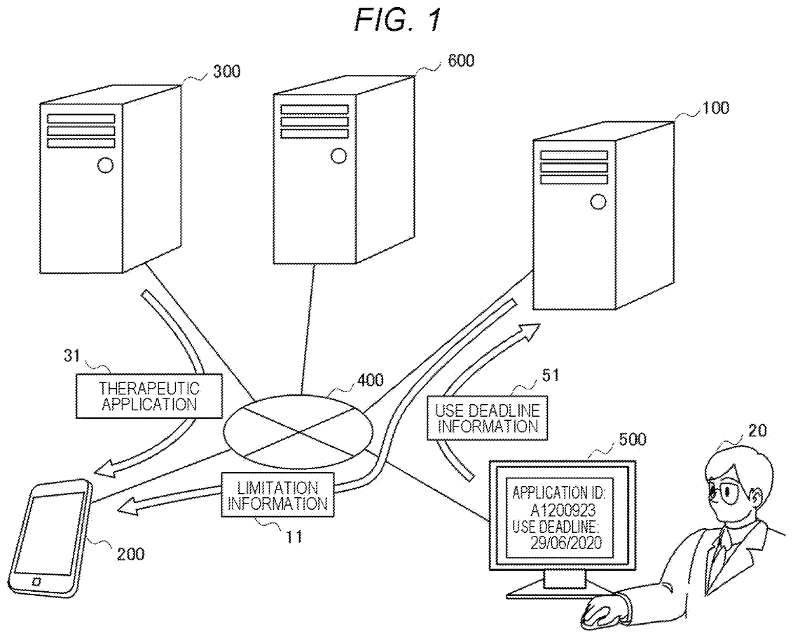

FIG. 1 is a schematic diagram schematically illustrating a therapeutic application management system 1 according to a first example and is a system diagram illustrating an example of a system configuration thereof. As illustrated in FIG. 1, the therapeutic application management system 1 includes a management server 100 and a delivery server 300 and may further include a user terminal 200, a medical worker terminal 500, and an operation server 600. The management server 100, the user terminal 200, the delivery server 300, the medical worker terminal 500, and the operation server 600 are communicatively connected to each other via a network 400. Only one for each device is illustrated in FIG. 1, but the number of terminals and servers is not limited to one, and may be two or more.

The management server 100 is a server device that manages a therapeutic application which is used by a patient with the user terminal 200 using the user terminal 200. The delivery server 300 is a server device having a function of delivering a therapeutic application to the user terminal 200. The user terminal 200 is an information processing terminal which is carried by a patient and is a so-called smartphone, a tablet terminal, a mobile phone, and a PC, for example. The medical worker terminal 500 is an information processing device that is used by a medical worker and is a so-called smartphone, a tablet terminal, a mobile phone, and a PC, for example, similarly to the user terminal 200. The operation server 600 is a server device that provides treatment information on treatment of a patient to the user terminal 200 of the patient when a therapeutic application is executed.

A therapeutic application is application software that manages daily conditions of a patient and performs guidance with medical knowledge personalized to the patient.

A medical worker is a person working in medicine and is, for example, a doctor, a nurse, a mentor, or a pharmaceutical chemist.

In the therapeutic application management system 1 according to this example, a medical worker 20 inputs a use deadline of a therapeutic application to the medical worker terminal 500. The input use deadline is transmitted as use deadline information 51 to the management server 100, and the management server 100 registers and manages the use deadline for each patient and for each therapeutic application. The use deadline information 51 may be set in advance in the management server 100 for each therapeutic application.

On the other hand, a patient downloads a therapeutic application 31 delivered from the delivery server 300 using the user terminal 200 and uses the therapeutic application. The management server 100 collects, stores, and manages various types of data which are stored by allowing the patient to use the therapeutic application. When the user terminal 200 executes the therapeutic application, treatment information which is provided from the management server 100 or the operation server 600 can be displayed as information on treatment of the patient of the user terminal 200. When the use deadline of the therapeutic application expires, limitation information 11 is transmitted to the user terminal 200 to limit use of the therapeutic application.

This will be described below in detail. A network 400 is a network is a network for connecting various devices to each other and may be, for example, a wireless network or a wired network. Specifically, the network is a wireless LAN (WLAN), a wide area network (WAN), integrated service digital networks (ISDNs), wireless LANs, Long Term Evolution (LTE), LTE-Advanced, 4-th Generation (4G), 5-th Generation (5G), Code Division Multiple Access (CDMA), WCDMA (registered trademark), or Ethernet (registered trademark). The network is not limited to these examples, and may be, for example, Public Switched Telephone Network (PSTN), Bluetooth (registered trademark), Bluetooth Low Energy (BLE), an optical network, Asymmetric Digital Subscriber Line (ADSL), a satellite communication network, or any network. The network may be referred to as a home network when it is provided in a home of a user. The network may be, for example, Narrow Band IoT (NB-IoT) or enhanced Machine Type Communication (eMTC). NB-IoT or eMTC is a wireless communication system dedicated for IoT and is a network enabling long-distance communication with low costs and low power consumption. The network may be a combination of the examples. The network may include a plurality of different networks in which the examples are combined. For example, the network may include a wireless network based on LTE and a wired network such as Intranet which is a closed network.

### Example of Configuration of Management Server 100

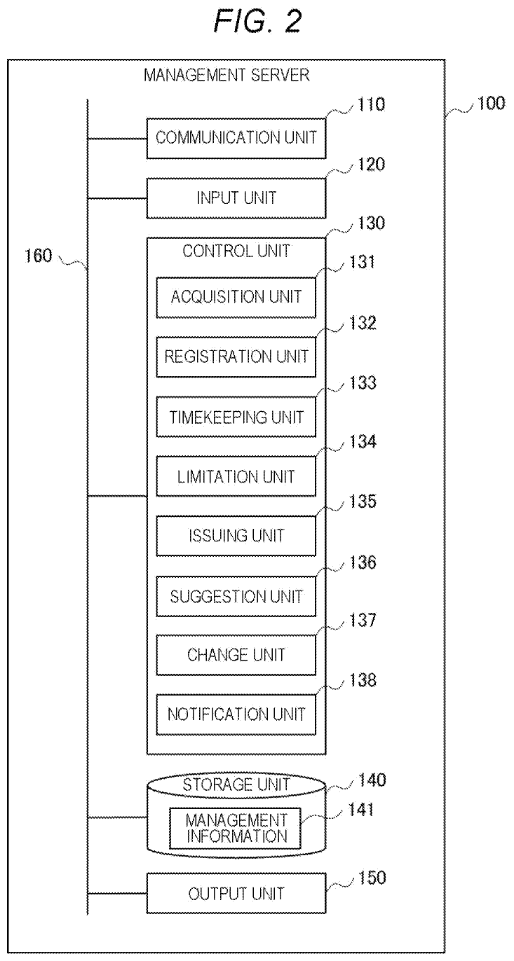

FIG. 2 is a block diagram illustrating an example of a configuration of the management server 100.

As illustrated in FIG. 2, the management server 100 includes a communication unit 110, an input unit 120, a control unit 130, a storage unit 140, and an output unit 150. The communication unit 110, the input unit 120, the control unit 130, the storage unit 140, and the output unit 150 may be configured to communicate with each other via a bus 160.

The communication unit 110 is a communication interface having a function of communicating with another device. The communication unit 110 may use any communication protocol for communication as long as it can communicate with another device and may perform any of wired communication and wireless communication. The communication unit 110 communicates with the user terminal 200, the delivery server 300, the medical worker terminal 500, or the operation server 600 in accordance with an instruction from the control unit 130. The communication unit 110 receives, for example, information indicating a therapeutic application, information on a patient who uses the therapeutic application, and information on a use deadline of the therapeutic application from the medical worker terminal 500 and delivers the received information to the control unit 130. For example, the communication unit 110 receives request information for requesting an activation code from the user terminal 200 and delivers the received request information to the control unit 130. For example, the communication unit 110 transmits an activation code or limitation information for limiting use of the therapeutic application to the user terminal 200 in accordance with an instruction from the control unit 130.

The input unit 120 is an input interface having a function of receiving an input from an operator of the management server 100, for example, and transmitting the received input to the control unit 130. The input unit 120 may be software keys of a touch panel, for example, or may be hardware keys. Alternatively, the input unit 120 may be a microphone for receiving a voice input. The input unit 120 receives an input from, for example, a medical worker or an operator of the management server 100 and delivers the input details to the control unit 130.

The storage unit 140 has a function of storing various programs and various types of data required for operating of the management server 100. The storage unit 140 may be, for example, a hard disc drive (HDD), a solid state drive (SSD), or a flash memory. The storage unit 140 stores management information 141. The management information 141 is information for managing a therapeutic application of each user. Details of the management information 141 will be described later.

The output unit 150 has a function of outputting designated information in accordance with an instruction from the control unit 130. The output from the output unit 150 may be output of one of an image signal and a voice signal. The output based on an image signal may be output to a monitor connected to the management server 100 (or provided in the management server 100). The output based on a voice signal may be output to a speaker connected to the management server 100 (or provided in the management server 100).

The control unit 130 is a processor having a function of controlling the constituent units of the management server 100. The control unit 130 may be a single core or may be multiple cores.

The control unit 130 includes an acquisition unit 131, a registration unit 132, a timekeeping unit 133, a limitation unit 134, an issuing unit 135, a suggestion unit 136, and a change unit 137 as functions which are the control unit 130.

The acquisition unit 131 acquires information indicating a therapeutic application, a patient ID for identifying a patient for which the therapeutic application is to be prescribed, and use deadline information which is information indicating a deadline to which the therapeutic application is able to be used by the patient. The information indicating a therapeutic application may be identification information for identifying the therapeutic application or may be an application itself. Acquisition by the acquisition unit 131 may be performed by receiving information which is transmitted from the medical worker terminal 500 by the communication unit 110 or by acquiring information which is directly input to the input unit 120 by a medical worker. This information may be individually acquired when a correlation therebetween can be recognized by the control unit 130.

The registration unit 132 registers the information acquired by the acquisition unit 131 in the management information 141 in correlation with each other. In the storage unit 140, the updated management information 141 is stored by the registration unit 132.

The timekeeping unit 133 counts a date and time. The timekeeping unit 133 may have a timekeeping function and count a date and time or may count a date and time by acquiring current date and time information from an external device via the communication unit 110.

The limitation unit 134 successively monitors the management information 141 and determines whether there is a therapeutic application of which the use deadline expires. When it is determined that there is a therapeutic application of which the use deadline expires, the limitation unit 134 identifies a patient using the therapeutic application using the management information 141. Then, the limitation unit 134 generates limitation information (a code) for limiting the therapeutic application used in the user terminal 200 of the identified patient and transmits the generated limitation information to the identified user terminal 200 via the communication unit 110. Accordingly, use of the therapeutic application used in the user terminal 200 of the patient is limited.

The issuing unit 135 issues an activation code for activating (validating, that is, enabling start of) each therapeutic application. The issuing unit 135 transmits the issued activation code to the user terminal 200 having requested the activation code via the communication unit 110.

The suggestion unit 136 calculates a use deadline of a therapeutic application which is prescribed for a patient based on treatment details received from a medical worker using a trained model having learned a relationship between treatment details and a use deadline to which the therapeutic application is able to be used. The suggestion unit 136 suggests the calculated use deadline to the medical worker. Suggestion to the medical worker may transmit the use deadline information to be suggested to the medical worker terminal 500 or may be displaying the use deadline information on a monitor connected to the management server 100 when the management server 100 is operated by a medical worker.

The change unit 137 receives a predetermined input and changes a use deadline of a therapeutic application in the management information 141. The predetermined input mentioned herein may be an input from an operator of the management server 100 having received an instruction from a medical worker, an input based on receiving of a change instruction of a medical worker from the medical worker terminal 500, or an input based on receiving of a change instruction from the user terminal 200 of a patient having received an instruction from a medical worker.

The notification unit 138 monitors the management information 141 and identifies a therapeutic application of which the use deadline is imminent. For example, when there is a therapeutic application of which the use deadline expires within a predetermined period (for example, two weeks), the notification unit 138 transmits information (a message) indicating that the use deadline of the therapeutic application is about to expire to the medical worker terminal 500 or the user terminal 200 via the communication unit 110. The information indicating that the use deadline of the therapeutic application is about to expire may include a message for suggesting extension of the use deadline.

An example of the configuration of the management server 100 is the same as described above.

### Example of Configuration of User Terminal 200

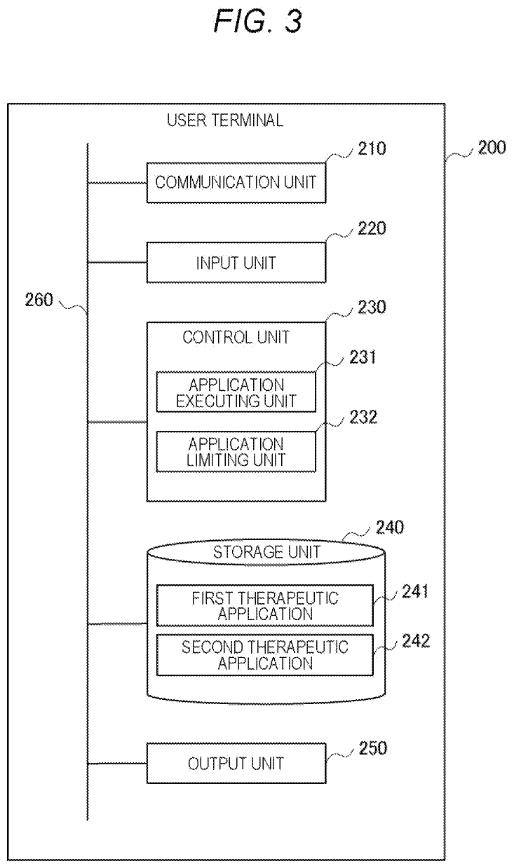

FIG. 3 is a block diagram illustrating an example of a configuration of the user terminal 200. The user terminal 200 includes a communication unit 210, an input unit 220, a control unit 230, a storage unit 240, and an output unit 250. The communication unit 210, the input unit 220, the control unit 230, the storage unit 240, and the output unit 250 may be configured to communicate with each other via a bus 260.

The communication unit 210 is a communication interface having a function of communicating with another device. The communication unit 210 may use any communication protocol for communication as long as it can communicate with another device and may perform any of wired communication and wireless communication. The communication unit 210 communicates with the management server 100 and the delivery server 300 in accordance with an instruction from the control unit 230.

The input unit 220 is an input interface having a function of receiving an input from a patient of the user terminal 200 and transmitting the received input to the control unit 230. The input unit 220 may be software keys of a touch panel, for example, or may be hardware keys. Alternatively, the input unit 220 may be a microphone for receiving a voice input. The input unit 220 delivers input details received from the patient to the control unit 230.

The control unit 230 is a processor having a function of controlling the constituent units of the user terminal 200. The control unit 230 may be a single core or may be multiple cores. The control unit 230 transmits request information for requesting a therapeutic application to the delivery server 300 via the communication unit 210 in response to an input from a patient.

The control unit 230 includes an application executing unit 231 and an application limiting unit 232 as functions which are the control unit 230.

The application executing unit 231 executes a therapeutic application downloaded from the delivery server 300. By executing the therapeutic application, the application executing unit 231 registers various types of information in the therapeutic application in response to an input from a patient, acquires information on treatment from the management server 100 or the operation server 600 storing treatment information, and displays the acquired information.

When limitation information 11 is received from the management server 100, the application limiting unit 232 limits use of the therapeutic application indicated by the limitation information 11. Limitation of use of a therapeutic application mentioned herein may be curbing start of the therapeutic application or limiting display of the therapeutic application even when it starts. Limiting of display mentioned herein may be limitation of display of all data which can be read using the therapeutic application or may be limitation of display of only some data thereof.

The storage unit 240 has a function of storing various programs and various types of data required for operating of the user terminal 200. The storage unit 240 may be, for example, an HDD, an SSD, or a flash memory. An example in which a first therapeutic application 241 and a second therapeutic application 242 are stored in the storage unit 240 is illustrated in FIG. 3.

The output unit 250 has a function of outputting designated information in accordance with an instruction from the control unit 230. The output from the output unit 250 may be output of one of an image signal and a voice signal. The output based on an image signal may be output to a monitor provided in the user terminal 200 (or connected to the user terminal 200). The output based on a voice signal may be output to a speaker provided in the user terminal 200 (or connected to the user terminal 200). The output unit 250 displays information which is provided by the therapeutic application when the application executing unit 231 executes the therapeutic application.

An example of the configuration of the user terminal 200 is the same as described above.

### Example of Configuration of Delivery Server 300

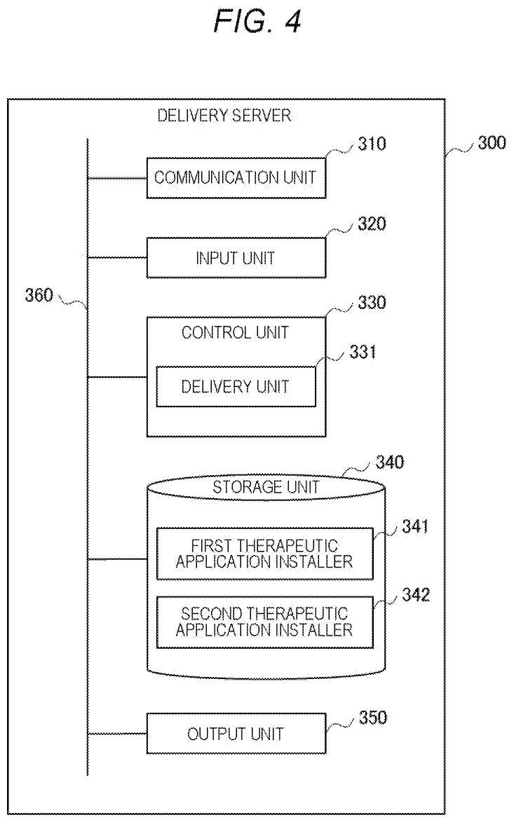

FIG. 4 is a block diagram illustrating an example of a configuration of the delivery server 300. The delivery server 300 is a download server that stores a therapeutic application designated by the management server 100 and delivers the therapeutic application in response to a request from the user terminal 200.

As illustrated in FIG. 4, the delivery server 300 includes a communication unit 310, an input unit 320, a control unit 330, a storage unit 340, and an output unit 350. The communication unit 310, the input unit 320, the control unit 330, the storage unit 340, and the output unit 350 may be configured to communicate with each other via a bus 360.

The communication unit 310 is a communication interface having a function of communicating with another device. The communication unit 310 may use any communication protocol for communication as long as it can communicate with another device and may perform any of wired communication and wireless communication. The communication unit 310 communicates with the management server 100 and the user terminal 200 in accordance with an instruction from the control unit 330.

The input unit 320 is an input interface having a function of receiving an input from an operator of the delivery server 300, for example, and transmitting the received input to the control unit 330. The input unit 320 may be software keys of a touch panel, for example, or may be hardware keys. Alternatively, the input unit 320 may be a microphone for receiving a voice input.

The control unit 330 is a processor having a function of controlling the constituent units of the delivery server 300. The control unit 330 may be a single core or may be multiple cores.

The control unit 330 includes a delivery unit 331 as a function which is the control unit 330.

The delivery unit 331 identifies an installer of a therapeutic application indicated by request information transmitted from the communication unit 310 and reads the identified installer from the storage unit 340. The delivery unit 331 transmits the read installer of the therapeutic application to the user terminal 200 having transmitted the request information via the communication unit 310.

The storage unit 340 has a function of storing various programs and various types of data required for operating of the delivery server 300. The storage unit 340 may be, for example, an HDD, an SSD, or a flash memory. The storage unit 340 stores (installers of) all therapeutic applications requested from the management server 100. An example in which a first therapeutic application installer 341 and a second therapeutic application installer 342 are stored is illustrated in FIG. 4.

The output unit 350 has a function of outputting designated information in accordance with an instruction from the control unit 330. The output from the output unit 350 may be output of one of an image signal and a voice signal. The output based on an image signal may be output to a monitor connected to the delivery server 300 (or provided in the delivery server 300). The output based on a voice signal may be output to a speaker provided in the delivery server 300 (or connected to the delivery server 300).

An example of the configuration of the delivery server 300 is the same as described above.

### Example of Configuration of Medical Worker Terminal 500

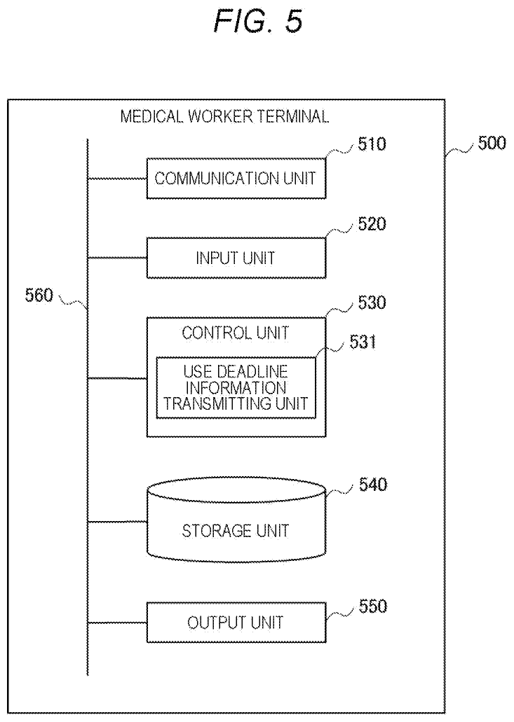

FIG. 5 is a block diagram illustrating an example of a configuration of the medical worker terminal 500. The medical worker terminal 500 is an information processing terminal that is used by a medical worker. The medical worker terminal 500 includes a communication unit 510, an input unit 520, a control unit 530, a storage unit 540, and an output unit 550. The communication unit 510, the input unit 520, the control unit 530, the storage unit 540, and the output unit 550 may be configured to communicate with each other via a bus 560.

The communication unit 510 is a communication interface having a function of communicating with another device. The communication unit 510 may use any communication protocol for communication as long as it can communicate with another device and may perform any of wired communication and wireless communication. The communication unit 510 communicates with the management server 100, the delivery server 300, and the operation server 600 in accordance with an instruction from the control unit 530.

The input unit 520 is an input interface having a function of receiving an input from a patient of the user terminal 200 and transmitting the received input to the control unit 530. The input unit 520 may be software keys of a touch panel, for example, or may be hardware keys. Alternatively, the input unit 520 may be a microphone for receiving a voice input. The input unit 520 delivers input details received from the patient to the control unit 530.

The input unit 520 receives an input of information indicating a use deadline of a therapeutic application from a medical worker and delivers the received input to the control unit 530. Specifically, the input unit 520 receives designation of an application ID for designating a therapeutic application of which a use deadline is set and a patient ID indicating a patient who uses the therapeutic application, receives an input of information indicating the use deadline of the designated therapeutic application, and delivers the received inputs to the control unit 530. The patient ID may not be input.

The control unit 530 is a processor having a function of controlling the constituent units of the medical worker terminal 500. The control unit 530 may be a single core or may be multiple cores.

The control unit 530 includes a use deadline information transmitting unit 531 which functions through the control unit 530.

The use deadline information transmitting unit 531 generates use deadline information in which the application ID input via the input unit 520 by the medical worker and the use deadline thereof are correlated. The use deadline information may additionally include a patient ID indicating a patient who is scheduled to use the therapeutic application. The use deadline information transmitting unit 531 transmits the generated use deadline information to the management server 100 via the communication unit 510.

The storage unit 540 has a function of storing various programs and various types of data required for operating of the medical worker terminal 500. The storage unit 540 may be, for example, an HDD, an SSD, or a flash memory.

The output unit 550 has a function of outputting designated information in accordance with an instruction from the control unit 530. The output from the output unit 550 may be output of one of an image signal and a voice signal. The output based on an image signal may be output to a monitor connected to the medical worker terminal 500 (or provided in the medical worker terminal 500). The output based on a voice signal may be output to a speaker provided in the medical worker terminal 500 (or connected to the medical worker terminal 500). The output unit 550 may display, for example, information indicating the use deadline of the therapeutic application input by the medical worker as image data.

An example of the configuration of the medical worker terminal 500 is the same as described above.

### Example of Configuration of Operation Server 600

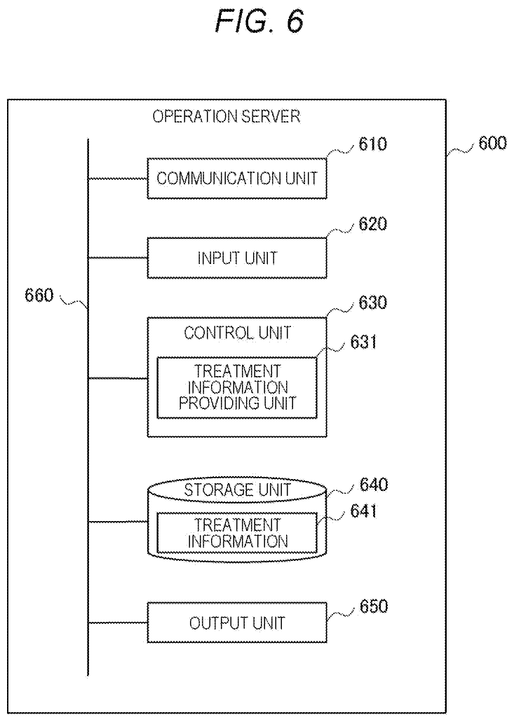

FIG. 6 is a block diagram illustrating an example of a configuration of the operation server 600. The operation server 600 is an information processing device (a server) that manages and operates information on treatment of a patient who uses a therapeutic application. The operation server 600 may be provided for each disease of patients or for each therapy facility (such as a hospital or a free dispensary) that treats patients, which is information on treatment of patients managed by the operation server 600, may be provided for each provider that provides therapeutic applications, or may be collectively managed by one server. As illustrated in FIG. 6, the operation server 600 includes a communication unit 610, an input unit 620, a control unit 630, a storage unit 640, and an output unit 650. The communication unit 610, the input unit 620, the control unit 630, the storage unit 640, and the output unit 650 may be configured to communicate with each other via a bus 560.

The communication unit 610 is a communication interface having a function of communicating with another device. The communication unit 610 may use any communication protocol for communication as long as it can communicate with another device and may perform any of wired communication and wireless communication. The communication unit 610 communicates with the management server 100 and the user terminal 200 in accordance with an instruction from the control unit 630.

The input unit 620 is an input interface having a function of receiving an input from a patient of the user terminal 200 and transmitting the received input to the control unit 630. The input unit 620 may be software keys of a touch panel, for example, or may be hardware keys. Alternatively, the input unit 620 may be a microphone for receiving a voice input. The input unit 620 delivers input details input from the patient to the control unit 630.

The input unit 620 receives an input of information on a patient to be examined from a medical worker (mainly a doctor) and delivers the received input to the control unit 630. Specifically, the information on a patient to be examined is not particular limited as long as it is information on the patient and may be information such as disease conditions of a patient, medicine prescribed for a patient, or a treatment progress from a medical worker. Specifically, the input unit 620 receives an input of a patient ID indicating a patient on which information has been input and information on the patient indicated by the patient ID and delivers the received inputs to the control unit 630.

The control unit 630 is a processor having a function of controlling the constituent units of the operation server 600. The control unit 630 may be a single core or may be multiple cores. When a patient ID and information on a patient are delivered from the input unit 620, the control unit 630 stores the information on a patient in correlation with the delivered patient ID as treatment information 641 in the storage unit 640.

The control unit 630 includes a treatment information providing unit 631 which functions through the control unit 630.

When a transmission request for treatment information is received from the user terminal 200 via the communication unit 610, the treatment information providing unit 631 identifies information on a patient correlated with the patient ID indicated by the transmission request for treatment information from the treatment information 641. The treatment information providing unit 631 transmits the identified treatment information (information on a patient) to the user terminal 200 via the communication unit 610.

The storage unit 640 has a function of storing various programs and various types of data required for operating of the operation server 600. The storage unit 640 may be, for example, an HDD, an SSD, or a flash memory. The storage unit 640 stores treatment information 641 on treatment of patients. In the treatment information, the information on patients is correlated with patient IDs of patients. The treatment information may be a so-called electronic medical record.

The output unit 650 has a function of outputting designated information in accordance with an instruction from the control unit 630. The output from the output unit 650 may be output of one of an image signal and a voice signal. The output based on an image signal may be output to a monitor connected to the operation server 600 (or provided in the operation server 600). The output based on a voice signal may be output to a speaker provided in the operation server 600 (or connected to the operation server 600). The output unit 650 may output, for example, the treatment information on patients.

An example of the configuration of the operation server 600 is the same as described above.

### Data

The management information 141 will be described below. As described above, the management information is information for allowing the management server 100 to manage therapeutic applications which are being used by patients.

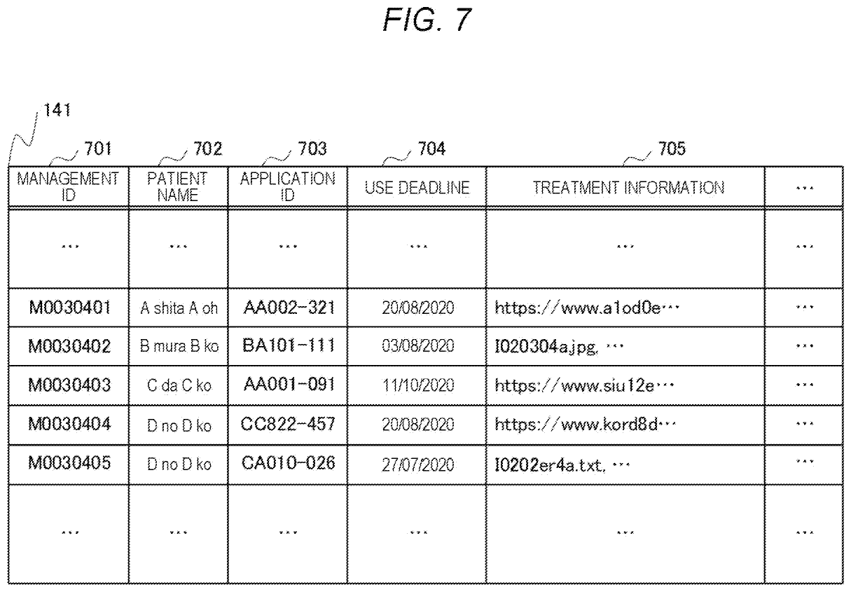

FIG. 7 is a conceptual diagram of data illustrating an example of a data structure of the management information 141. The management information 141 is information in which a management ID 701, a patient name 602, an application ID 703, a use deadline 704, and treatment information 705 are correlated.

The management ID 701 is identification information for management which is conveniently assigned by the management server 100 to manage therapeutic applications which are used by patients included in the management information 141.

The patient name 602 is information indicating names of patients who use the therapeutic application. The patient name 602 may be an identifier for uniquely identifying a patient instead of the name of the patient.

The application ID 703 is identification information for uniquely identifying the therapeutic application which is used by a patient indicated by the corresponding the patient name 602.

The use deadline 704 is information indicating a use deadline of the therapeutic application indicated by the corresponding application ID 703. The use deadline indicated by the use deadline 704 is a deadline which is determined by a medical worker. The use deadline 704 indicates information of a date at which the use deadline expires, and this may be expressed by the number of days in which the corresponding therapeutic application is able to be used.

The treatment information 705 is information indicating actual data of information on treatment which can be ascertained using a therapeutic application used by a patient indicated by the corresponding patient name 602 and indicated by the application ID 703 or an address of a device that can ascertain the actual data. The device which can ascertain the actual data mentioned herein is a server device that stores information such as examination information in a medical institution in which the patient is actually treated and is a server device of a medical institution cooperating with the management server 100. When all the treatment information 705 is operated and managed by the operation server 600, it may not be registered in the management information 141. The treatment information 705 and the treatment information 641 stored in the operation server 600 may be the same or different. In an example in which both pieces of treatment information are different, information of smoking cessation treatment of a patient is stored in the management server 100 and information of diabetes treatment is stored in the operation server 600.

When a patient uses different therapeutic applications, the therapeutic applications are individually registered in the management information 141. In addition to such information, information such as addresses of the user terminals 200 of the patients or phone numbers which are contact addresses may be stored in the management information 141.

### Operations

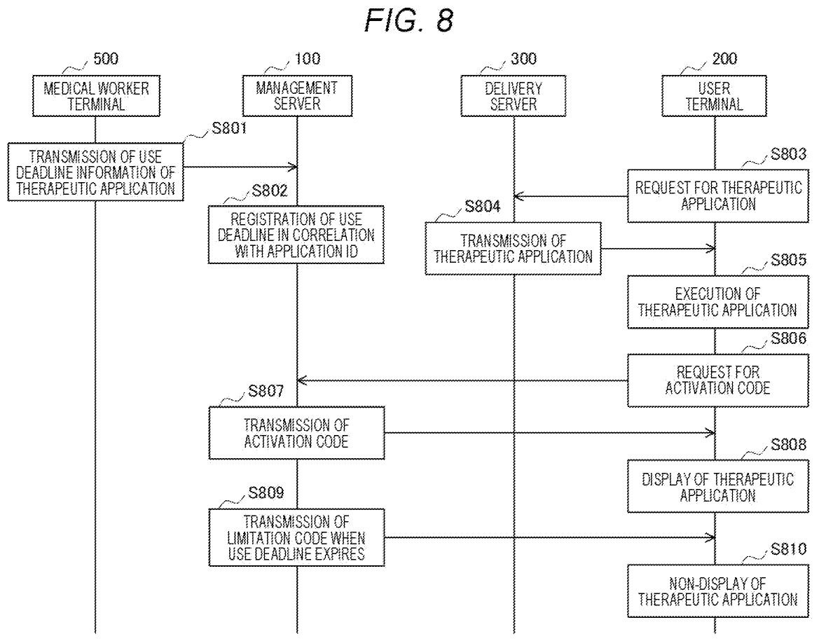

FIG. 8 is a sequence diagram illustrating an example of transmission and reception between devices in the therapeutic application management system 1.

As illustrated in FIG. 8, the medical worker terminal 500 transmits use deadline information of a therapeutic application input by a medical worker to the management server 100 (Step S801). In the use deadline information, a use deadline of a therapeutic application which is a target of the use deadline may be set by each type of application or may be individually set depending on conditions of the patients.

The management server 100 registers the use deadline included in the use deadline information for the therapeutic application indicated by the application ID included in the use deadline information received from the medical worker terminal 500 (Step S802).

On the other hand, the user terminal 200 transmits request information for requesting a therapeutic application to the delivery server 300 in response to an input from a patient of the user terminal 200 (Step S803).

The delivery server 300 receives the request information from the user terminal 200 and transmits the therapeutic application designated by the request information to the user terminal 200 (Step S804).

When the therapeutic application is received from the delivery server 300, the user terminal 200 executes the therapeutic application in accordance with an instruction from the patient (Step S805). When the therapeutic application is executed, the user terminal 200 transmits a request for an activation code for executing the therapeutic application to the management server 100 (Step S806).

The management server 100 receives the request for an activation code from the user terminal 200, ascertains whether a medical worker has permitted, and transmits the activation code (Step S807).

The patient of the user terminal 200 ascertains the activation code transmitted from the management server 100 and inputs the activation code. The therapeutic application to which the activation code has been input displays contents associated with treatment (Step S808). The contents associated with treatment displayed herein may be acquired from the treatment information 705 stored in the management server 100 or may be acquired from a predetermined operation server 600.

The management server 100 successively ascertains the use deadlines of the therapeutic applications and transmits limitation information (a code) for limiting the therapeutic application to the user terminal 200 of the patient using the therapeutic application when there is a therapeutic application of which the use deadline expires (Step S809). After the limitation information is received, the user terminal 200 does not display (deactivates) the therapeutic application indicated by the limitation information (Step S810). Accordingly, it is possible to limit use of a therapeutic application in the user terminal 200 and to prevent a patient who does not need to use the therapeutic application any more from misusing the therapeutic application.

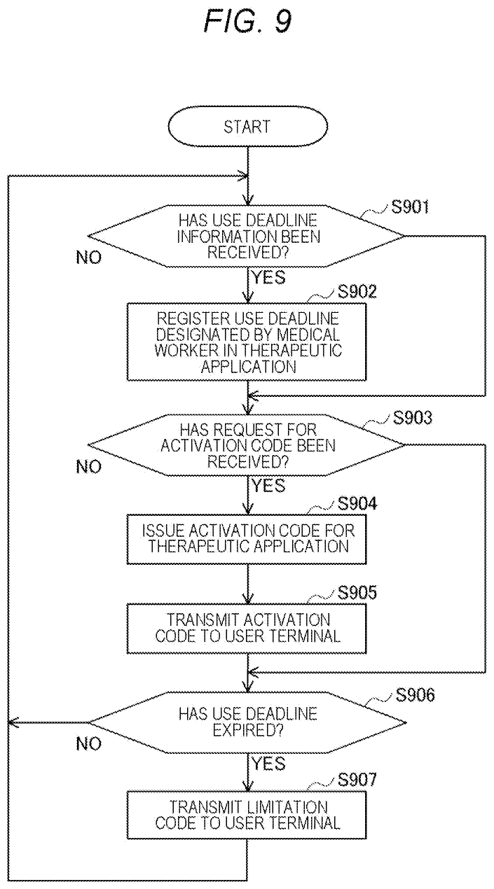

FIG. 9 is a flowchart illustrating an example of operations of the management server 100 for the transmission and reception illustrated in FIG. 8.

When a signal is received, the communication unit 110 of the management server 100 ascertains whether the signal is use deadline information. When use deadline information is received (YES in Step S901), the communication unit 110 delivers the received use deadline information to the control unit 130. When the received signal is not use deadline information (NO in Step S901), the routine proceeds to the process of Step S903.

When the use deadline information is delivered, the control unit 130 extracts an application ID and a use deadline of the therapeutic application included in the use deadline information. At this time, when a patient ID is included in the use deadline information, the patient ID is also extracted. Then, the registration unit 132 registers the use deadline in the management information 141 in correlation with the extracted application ID and the extracted patient ID (Step S902). Accordingly, the use deadline of the corresponding therapeutic application is managed in the management server 100.

When a signal is received, the communication unit 110 ascertains whether the signal is a signal for requesting an activation code of a therapeutic application. When a signal for requesting an activation code is received (YES in Step S903), the communication unit 110 delivers the received signal to the control unit 130. When a signal for requesting an activation code is not received (NO in Step S903), the routine proceeds to the process of Step S906.

When a request for an activation code is received, the control unit 130 identifies an application ID indicating the therapeutic application and a patient ID included in the request. The issuing unit 135 of the control unit 130 issues (generates) an activation code for the identified therapeutic application (Step S904). Then, the issuing unit 135 transmits the issued activation code to the user terminal 200 of the identified patient ID via the communication unit 110 (Step S905).

The limitation unit 134 of the control unit 130 normally (or appropriately, for example, once a day) monitors the management information 141 and compares a date and time counted by the timekeeping unit 133 with the use deadlines 704 of the therapeutic applications stored in the management information 141. That is, the limitation unit 134 determines whether a use deadline of each therapeutic application managed in the management information 141 expires (Step S906). When there is no therapeutic application of which the use deadline expires (NO in Step S906), the routine proceeds to the process of Step S901.

When there is a therapeutic application in which the use deadline expires, that is, the date and time counted by the timekeeping unit 133 passes beyond the use deadline 704 (YES in Step S906), the limitation unit 134 generates a limitation code for limiting start (or display) of the therapeutic application. Then, the limitation unit 134 transmits the generated limitation code to the user terminal 200 of the patient using the identified therapeutic application via the communication unit 110 (Step S907), and the routine returns to the process of Step S901. Accordingly, it is possible to limit use of a therapeutic application of which the use deadline expires in the user terminal 200.

The processes of Steps S901 and S902, the processes of Steps S903 to S905, and the processes of Steps S906 and S907 may be performed in parallel.

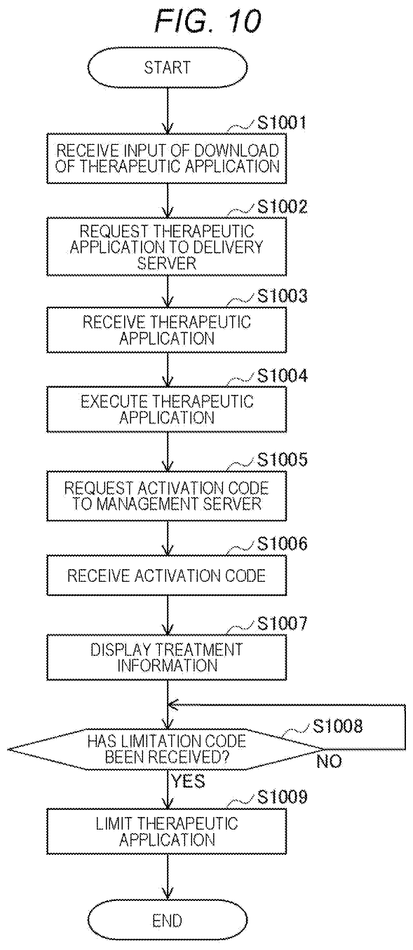

FIG. 10 is a flowchart illustrating an example of operations of the user terminal 200 for the transmission and reception illustrated in FIG. 8.

The input unit 220 of the user terminal 200 receives an input for instructing to download a therapeutic application from a patient (Step S1001). The input unit 220 delivers the received input details to the control unit 230. The control unit 230 transmits application request information for requesting the designated therapeutic application to the delivery server 300 storing the therapeutic application via the communication unit 210 based on the delivered input details (Step S1002). Accordingly, an installer (which may be an execution program) of the designated therapeutic application is transmitted from the delivery server 300.

The communication unit 210 receives the installer of the therapeutic application transmitted from the delivery server 300 (Step S1003). The communication unit 210 delivers the received installer of the therapeutic application to the control unit 230. When the installer of the therapeutic application is delivered, the control unit 230 installs the therapeutic application, and the application executing unit 231 executes the therapeutic application (Step S1004).

When the therapeutic application is executed at the first time, the application executing unit 231 transmits a request for an activation code to the management server 100. The activation code may be a code of which an input is required whenever the therapeutic application is executed. The application executing unit 231 transmits code request information for requesting the activation code to the management server 100 via the communication unit 210 (Step S1005). Accordingly, the management server 100 issues and transmits the activation code to the user terminal 200.

The communication unit 210 receives the activation code of the therapeutic application transmitted from the management server 100 (Step S1006). The communication unit 210 delivers the received activation code to the control unit 230. Then, the control unit 230 causes the output unit 250 to output the delivered activation code. That is, the activation code is displayed on a monitor of the user terminal 200. Then, when the patient inputs the displayed activation code to the input unit 220, the therapeutic application is executed. Inputting of the activation code may be directly input to the therapeutic application at the same time as reception instead of input from the patient.

The therapeutic application is activated with receiving the input of the activation code, and the application executing unit 231 displays treatment information requested with the input from the patient (Step S1007). At this time, details input to the therapeutic application by the patient (for example, opinion on treatment or behavior details on treatment) may be uploaded to the management server 100 or a server that issues the therapeutic application and storing treatment information of the patient.

The application limiting unit 232 of the control unit 230 ascertains whether a limitation code of the therapeutic application is received from the management server 100 (Step S1008). When a limitation code is received from the management server 100, the communication unit 210 delivers the limitation code to the control unit 230. When the limitation code is delivered, the application limiting unit 232 of the control unit 230 extracts an application ID included in the limitation code. Then, the application limiting unit 232 limits the therapeutic application corresponding to the extracted application ID (Step S1009). Accordingly, it is possible to limit use of the therapeutic application of which the use deadline determined by a medical worker expires.

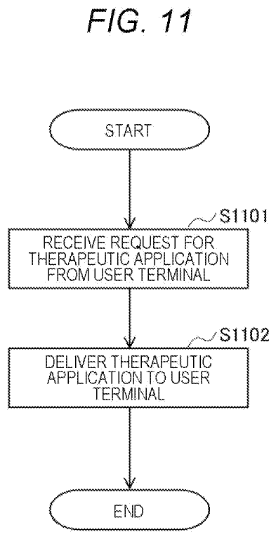

FIG. 11 is a flowchart illustrating an example of operations of the delivery server 300 for the transmission and reception illustrated in FIG. 8.

As illustrated in FIG. 11, the communication unit 310 of the delivery server 300 receives a request signal for requesting a therapeutic application from the user terminal 200 (Step S1101). The communication unit 310 delivers the received request signal to the control unit 330.

When the request signal is delivered, the delivery unit 331 of the control unit 330 extracts an application ID indicating a therapeutic application which is desired by a patient and which is included in the delivered request signal. Then, the delivery unit 331 acquires an installer of the therapeutic application indicated by the extracted application ID from the storage unit 340. The delivery unit 331 delivers the acquired installer of the therapeutic application to the user terminal 200 having transmitted the request signal via the communication unit 310 (Step S1102). Accordingly, the patient can acquire the therapeutic application.

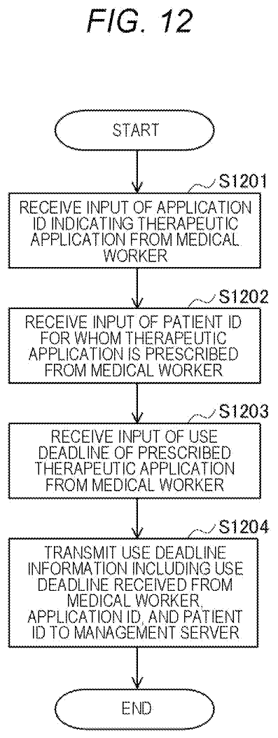

FIG. 12 is a flowchart illustrating an example of operations of the medical worker terminal 500 for the transmission and reception illustrated in FIG. 8.

As illustrated in FIG. 12, the input unit 520 of the medical worker terminal 500 receives an input of an application ID indicating a therapeutic application from a medical worker (Step S1201). The input unit 520 delivers the received application ID to the control unit 530.

The input unit 520 receives an input of a patient ID indicating a patient for whom a therapeutic application indicated by the application ID received in Step S1201 is prescribed from the medical worker (Step S1202). The input unit 520 delivers the received patient ID to the control unit 530. When the received input of a use deadline is not for any identified patient but is uniformly set for one type of therapeutic applications, inputting of a patient ID may be skipped. When the patient ID is skipped, the input unit 520 delivers information indicating that there is no patient ID to the control unit 530.

The input unit 520 receives a use deadline in which the therapeutic application indicated by the application ID received in Step S1201 is able to be used from the medical worker (Step S1203). The use deadline may be information indicating the last day to which the therapeutic application is able to be used or may be information indicating a period in which the therapeutic application is able to be used. The number of days until the use deadline may differ depending on patients in spite of the same therapeutic application. The input unit 520 delivers the received use deadline to the control unit 530.

The use deadline information transmitting unit 531 of the control unit 530 generates use deadline information including the delivered application ID, a patient ID (which is omitted unless there is a patient ID), and the use deadline. The use deadline information transmitting unit 531 transmits the generated use deadline information to the management server 100 via the communication unit 510 (Step S1204), and the routine ends.

Accordingly, the management server 100 can manage therapeutic applications used by patients using the use deadline designated by a medical worker.

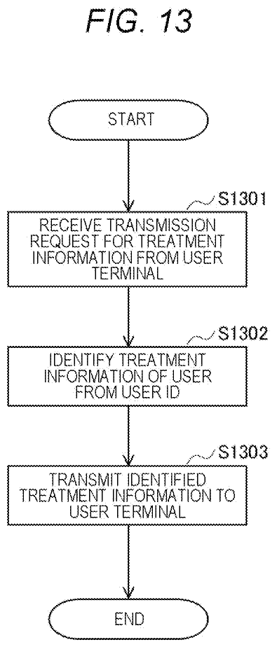

FIG. 13 is a flowchart illustrating an example of operations of the operation server 600. The communication unit 610 of the operation server 600 receives a transmission request for treatment information transmitted from a user terminal (Step S1301). The transmission request may include at least a user ID for identifying a user and include a password for browsing the treatment information in addition. When a transmission request for treatment information is received, the communication unit 610 delivers the transmission request to the control unit 630.

The treatment information providing unit 631 of the control unit 630 identifies a user ID from the delivered transmission request for treatment information. Then, the treatment information providing unit 631 identifies treatment information 641 corresponding to the identified user ID from the storage unit 640 (Step S1302). Then, the treatment information providing unit 631 transmits the identified treatment information to the user terminal 200 via the communication unit 610 (Step S1303).

Accordingly, the operation server 600 can provide information on treatment of a user to the user terminal 200. When a password is included in the transmission request for treatment information from the user terminal 200, authentication of the password is first performed in Step S1302 and identification of treatment information can be performed when the password is authenticated.

### CONCLUSION

As described above in the example, the management server 100 manages a therapeutic application using a use deadline in which the therapeutic application is able to be used. Since the therapeutic application includes information associated with treatment of a patient and can enable reading the information, it is preferable that the patient be prevented from misusing the therapeutic application due to continuous and free use of the therapeutic application. Therefore, according to the example, by managing a therapeutic application according to a use deadline designated by a medical worker, it is possible to prevent such misuse. It is also possible to improve reliability of a therapeutic application because the use deadline thereof is set by a medical worker.

### Supplementation

The therapeutic application management system according to the example is not limited to the configurations described in the example. Other modified examples and detailed examples will be described below.

(1) In the aforementioned example, execution of a therapeutic application of which a use deadline has expired is limited by transmitting limitation information (a code) for limiting use of the therapeutic application from the management server 100 to the user terminal 200 that executes the therapeutic application, but this disclosure is not limited thereto. Limitation of use of a therapeutic application may use other methods.

For example, a patient may be requested to input a password whenever the patient uses a therapeutic application, and use of a therapeutic application may be limited by limiting access to the therapeutic application when a date input at which the password has been input passes beyond the use deadline of the therapeutic application. Specifically, the user terminal 200 executes a therapeutic application in response to an input from the patient. When the therapeutic application starts, the therapeutic application requests the patient to input a password for the therapeutic application. That is, the user terminal 200 displays a UI screen for inputting a password for starting the therapeutic application. The patient inputs the notified password to the UI screen. Then, the user terminal 200 transmits the password input by the patient to the management server 100. The management server 100 ascertains whether the use deadline of the therapeutic application of the patient has expired with reference to the management information 141 at the time of authentication of the received password. When the use deadline has expired, the management server 100 notifies the user terminal 200 that the use deadline has expired and thus the therapeutic application cannot be executed. The user terminal 200 notifies the patient that the therapeutic application cannot be used and a reason thereof by displaying the notification. In this way, use of the therapeutic application of which the use deadline set by a medical worker has expired may be limited.

The password may be a one-time password or may be a password transmitted from the management server 100 to the user terminal 200 whenever the therapeutic application is used. In this example, when the use deadline for using the therapeutic application has expired, the management server 100 may limit the patient's use of the therapeutic application by not issuing the one-time password.

Limitation of use of a therapeutic application may be performed by the therapeutic application itself. That is, a therapeutic application may be delivered from the delivery server 300 in a state in which use deadline information has been embedded in the therapeutic application. The user terminal 200 requests the delivery server 300 to deliver a therapeutic application. At this time, the delivery server 300 transmits an installer of the therapeutic application in which information of a use deadline set by a medical worker (a use deadline which is set in advance by a medical worker of the medical worker terminal 500 or a use deadline which is input by a medical worker at the time of issuing the request) is embedded to the user terminal 200. When a patient starts the therapeutic application (for example, once a day), the user terminal 200 acquires information of the start date and compares the acquired date with the embedded use deadline. When the acquired date has passed beyond the use deadline, the user terminal 200 does not start the therapeutic application. In this way, start of the therapeutic application may be limited by the user terminal 200 and the therapeutic application.

Alternatively, an activation code may be operated like a so-called electronic certificate. That is, use of a therapeutic application may be limited by adding a use deadline to an activation code, enabling access to the management server 100 or a server storing treatment information when the use deadline has not expired at the time of execution of the therapeutic application, and disabling access to the management server 100 or the server storing treatment information when the use deadline has expired.

Alternatively, use of a therapeutic application may be limited by the operation server 600. Limitation of use of a therapeutic application using the operation server 600 may be one of the aforementioned aspects. For example, the aspect in which the operation server 600 transmits limitation information to the user terminal 200 may be employed. When a password is input whenever a user accesses the treatment information 641 using the user terminal 200, use of the therapeutic application may be limited by not providing treatment information when the use deadline set for the therapeutic application has expired.

(2) In the aforementioned example, the management server 100 performs only management of therapeutic applications, but may additionally have a function of the delivery server 300. That is, the management server 100 may have a function of delivering a therapeutic application. In this example, the delivery server 300 does not need to be provided in the therapeutic application management system 1.

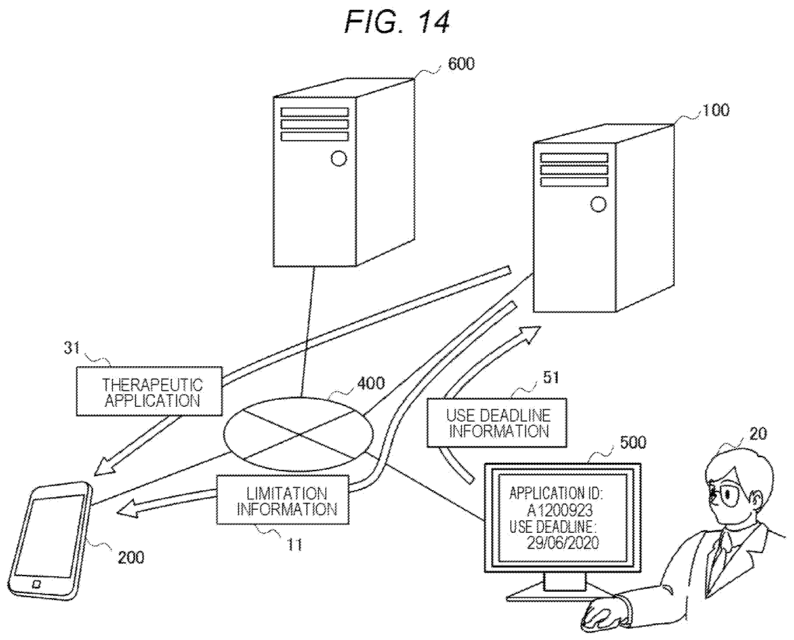

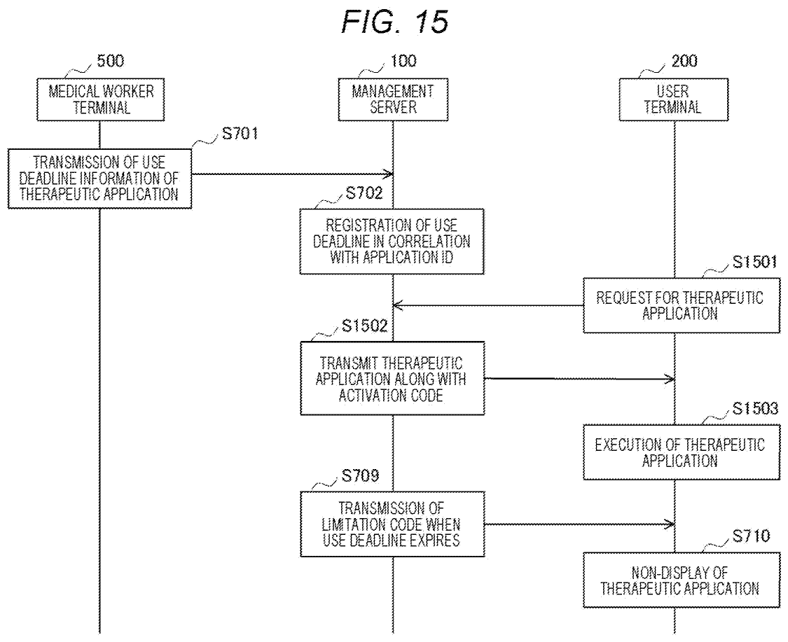

An example of a system configuration in this example is illustrated in FIG. 14. FIG. 15 illustrates an example of transmission and reception between devices when the transmission and reception illustrated in FIG. 8 is changed to this modified example.

As illustrated in FIG. 14, a therapeutic application management system includes a management server 100 and may further include a user terminal 200, a medical worker terminal 500, and an operation server 600. The devices are connected to each other to communicate with each other via a network 400. In the example illustrated in FIG. 14, the management server 100 also has the function of the delivery server 300 according to the example. That is, the control unit 130 of the management server 100 has the function of the delivery unit 311 of the delivery server 300.

FIG. 15 illustrates an example of transmission and reception between the devices in the therapeutic application management system illustrated in FIG. 14. In the sequence diagram illustrated in FIG. 15, a request destination to which the user terminal 200 first requests a therapeutic application is the management server 100. Processes different from those in the sequence diagram illustrated in FIG. 8 will be described below, the same processes as those in the sequence diagram illustrated in FIG. 8 will be referred to by the same reference signs, and description thereof will be omitted.

As illustrated in FIG. 15, the user terminal 200 requests the management server 100 to transmit a therapeutic application (Step S1501).

Accordingly, the management server 100 identifies a designated therapeutic application. Then, the management server 100 generates an activation code for the identified therapeutic application. The management server 100 transmits the therapeutic application along with the generated activation code to the user terminal 200 (Step S1502).

When the therapeutic application is received, the user terminal 200 executes the therapeutic application in response to an input from a patient of the user terminal 200 (Step S1503). At this time, the user terminal 200 receives the activation code already and thus can execute the therapeutic application immediately. In this way, when a therapeutic application is delivered from the management server 100, transmission of a request for an activation code from the user terminal 200 and transmission of an activation code from the management server 100 in response to the request can be skipped. As in the example illustrated in FIG. 8, the management server 100 may transmit only an installer of the therapeutic application when the therapeutic application is transmitted to the user terminal 200 and the user terminal 200 may request an activation code at the time of execution of the therapeutic application.

In this way, the management server 100 may deliver a therapeutic application to the user terminal 200.

(3) Details of the suggestion unit 136 in the aforementioned example will be described below. The suggestion unit 136 is for suggesting a use deadline of a therapeutic application as described above and is validated particularly when a use deadline is set for each patient, but the suggestion unit can also be applied to a configuration in which a use deadline is set for a therapeutic application regardless of a patient using the therapeutic application.

The suggestion unit 136 may suggest a use deadline, for example, using a trained model having learned a relationship between disease conditions and the numbers of days of a use deadline set for the disease conditions by a medical worker in the past. At this time, the trained model may be a model having additionally learned attributes of patients to be prescribed (for example, information such as sex, age, or characters). When there is a therapeutic application of which a use deadline is to be set, the suggestion unit 136 inputs conditions of a disease to be treated in the therapeutic application and attributes of a patient to be treated and estimates the use deadline thereof. Then, the suggestion unit 136 transmits the estimated use deadline to the medical worker terminal 500 via the communication unit 110. By causing the suggestion unit 136 to suggest the use deadline to a medical worker in this way, the medical worker can set the use deadline by only approving or changing the use deadline and thus it is possible to reduce a burden on a medical worker.

The suggestion unit 136 may suggest a use deadline according to a predetermined criterion. That is, an initial value of a basic number of days in which a therapeutic application is able to be used is set in advance and the number of days which is to be added to or subtracted from the initial value for each disease condition and for each patient attribute is set. These settings may be stored in the storage unit 140. The suggestion unit 136 estimates the number of days which is to be added to or subtracted corresponding to a disease condition or an attribute based on information of the disease condition or an attribute of a patient to be prescribed which is input from a medical worker, an operator of the management server 100, for example, adds the estimated number of days to the initial value, and calculates the use deadline. The suggestion unit 136 may suggest the use deadline calculated in this way to a medical worker (the medical worker terminal 500).

For example, by setting a use deadline of a therapeutic application to be shorter such that a patient with an advanced disease can be finely examined by a medical worker, the medical worker can easily contact the patient to update the number of days until the use deadline. For example, since it is assumed that an industrious patient industriously performs a daily input to a therapeutic application, it is possible that a medical worker can easily manage a disease condition of the patient and thus the use deadline is set to be longer. The suggestion unit 136 can suggest the use deadline of the therapeutic application by learning such cases.

(4) Details of the change unit 137 and the notification unit 138 in the aforementioned example will be described below.

For example, the change unit 137 changes a use deadline of a therapeutic application when change information instructing to change the use deadline of the therapeutic application is received from the medical worker terminal 500 by the communication unit 110. Changing of a use deadline of a therapeutic application may be changing of a use deadline of a therapeutic application of one patient or may be changing of use deadlines of the same types of therapeutic applications. Changing a use deadline mentioned herein may be shortening the use deadline or extending the use deadline.

Alternatively, when request information for extending a use deadline is received from the user terminal 200, the change unit 137 transmits a request indicated by the request information and request information for designation of a use deadline when the use deadline is extended to the medical worker terminal 500 via the communication unit 110. Then, when a medical worker's approval is issued from the medical worker terminal 500, the change unit 137 changes the use deadline 704 of the corresponding therapeutic application to the designated use deadline.

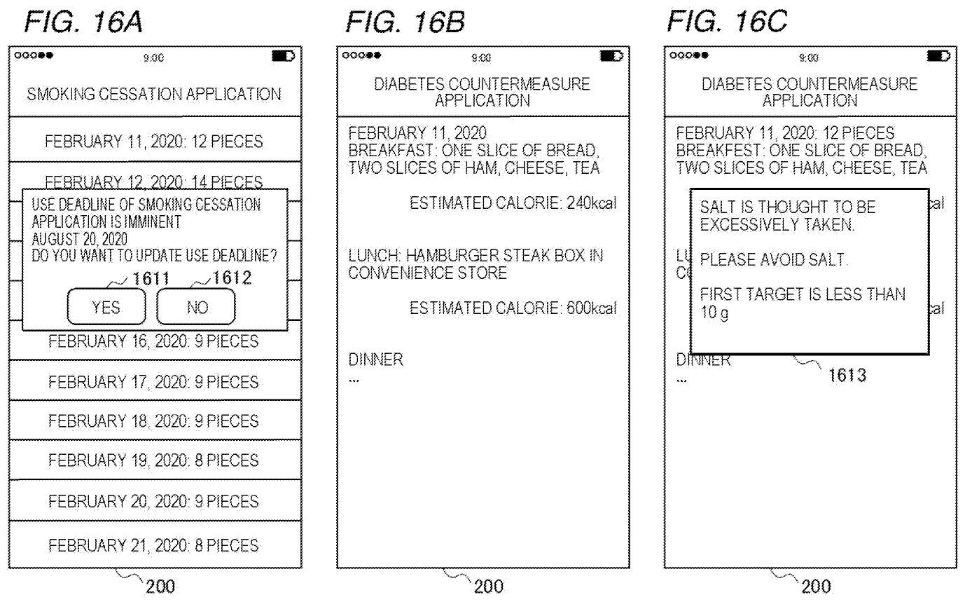

As a trigger for changing performed by the change unit 137, the notification unit 138 notifies the patient of the user terminal 200 or the medical worker of the medical worker terminal 500 that the use deadline of the therapeutic application is imminent. FIG. 16A illustrates an example of a notification which is displayed on the user terminal 200. The user terminal 200 displays a message for notifying that the use deadline of the therapeutic application is imminent when the application executing unit 231 executes the therapeutic application and when a notification indicating that the use deadline of the therapeutic application is imminent is received from the notification unit 138 of the management server 100. At this time, the message may display buttons 1611 and 1612 for inputting whether to receive an input for requesting update of the use deadline as illustrated in FIG. 16A. When an input to the button 1611 for approving update of the use deadline is performed by a patient, the application executing unit 231 transmits the aforementioned request information to the management server 100 via the communication unit 210.

An example in which the notification unit 138 notifies the user terminal 200 has been described above, but when the therapeutic application which is being executed in the user terminal 200 stores information of the use deadline, the therapeutic application may compare the use deadline with a current date and time and perform notification when the use deadline is imminent.

In addition to notifying that the use deadline is imminent at a timing at which the use deadline will expire in a predetermined number of days, the notification unit 138 may notify of the use deadline when the use deadline is inquired of from the user terminal 200 or the operation server 600. Specifically, when a patient inputs an inquiry about the use deadline of the therapeutic application to the input unit 220 of the user terminal 200, the user terminal 200 transmits deadline inquiry information to the management server 100. The deadline inquiry information includes an application ID indicating a therapeutic application of which the use deadline is inquired of. Similarly, when an operator inputs an inquiry about the use deadline of the therapeutic application to the input unit 620 of the operation server 600, the operation server 600 transmits deadline inquiry information including the application ID to the management server 100. When the deadline inquiry information is received, the notification unit 138 of the management server 100 identifies the use deadline 704 correlated with the application ID indicated by the deadline inquiry information and transmits information of the identified use deadline to the device having transmitted an inquiry. In this way, the notification unit 138 may notify of the use deadline in response to an inquiry from the user terminal 200 or the operation server 600.

(5) A display example of a therapeutic application in the aforementioned example is illustrated in FIGS. 16A to 16C. FIG. 16A illustrates an example of a use deadline which is imminent as described above. FIG. 16B illustrates a display example of a therapeutic application for diabetes as an example of a therapeutic application.

In diabetes, management of an intake calorie in each meal of a patient is an important item. Therefore, a patient may record details of each meal using a therapeutic application, the therapeutic application can record details of the meal and display a list of details of the meals as illustrated in FIG. 16B. The therapeutic application may estimate an intake calorie from the input details and display the estimated calorie. The estimated calorie may be displayed in text, but the estimated calorie may be displayed using an image. The therapeutic application may perform suggestion of treatment for a patient based on the intake details. FIG. 16C illustrates a screen view of an example of such suggestion. As illustrated in FIG. 16C, the therapeutic application may display a message 1613 suggesting improvement of a patient with reference to the input details, actions of the patient up to now, treatment records, and the like.

Diabetes is described as an example, but the therapeutic application may be associated with certain treatment, and may be, for example, a therapeutic application for smoking cessation, a therapeutic application for alcohol abstinence, or a therapeutic application for dementia. The therapeutic application may include different details or common details depending on diseases.

(6) In the aforementioned example, the therapeutic application management method is performed by the management server 100 and causes the processor of the management server 100 to execute a management program, for example, but it may be logical circuits (hardware) or dedicated circuits which are formed of integrated circuits (an integrated circuit (IC) chip or a large scale integration (LSI) chip), for example, in a device. Such circuits may be one or more integrated circuits, and the functions of a plurality of functional units described above in the example may be one integrated circuit. LSI may be referred to as VLSI, super LSI, and ultra LSI, for example, according to a difference in a degree of integration.

The management program may be recorded on a recording medium which can be read by a processor, and a “non-transitory tangible medium” such as a tape, a disk, a card, a semiconductor memory, or a programmable logic circuit can be used as the recording medium. The management program may be supplied to the processor via an arbitrary transmission medium (such as communication networks or broadcast waves) that can transmit the management program. That is, for example, a configuration in which the management program is downloaded from a network and executed by an information processing device such as a smartphone may be employed. This program may be in the form of a data signal embedded in carrier waves and electronic transmission of the management program.

The management program may be mounted, for example, using a script language such as ActionScript or JavaScript (registered trademark) or an object-oriented programming language such as Objective-C, Java (registered trademark), C++, Python, or R.

(7) Various examples described above in the example or various examples described in the “Supplement” may be appropriately combined. The operations described in the flowcharts may be changed in the execution order or performed in parallel unless conflictions arise as a result.

1. A therapeutic application management system comprising:
an acquisition unit configured to acquire an application ID indicating a therapeutic application prescribed for a patient by a medical worker, a patient ID that identifies a patient for whom the therapeutic application is to be prescribed, and use deadline information indicating a deadline to which the therapeutic application is able to be used;
a registration unit configured to register the application ID that identifies the therapeutic application, the patient ID, and the use deadline information in a storage unit in correlation with each other;
a timekeeping unit configured to count date and time information; and
a limitation unit configured to limit use of the therapeutic application transmitted to a terminal of the patient when the date and time information counted by the timekeeping unit has passed beyond a date and time determined by the use deadline information.

2. The therapeutic application management system according to claim 1, wherein the use deadline information is a deadline determined by the medical worker.

3. The therapeutic application management system according to claim 1, wherein the use deadline information is information in which the number of days in which the therapeutic application is able to be used after the therapeutic application has been prescribed is defined.

4. The therapeutic application management system according to claim 1, further comprising:
a reception unit configured to receive request information that requests an activation code to validate the therapeutic application downloaded by the patient using the terminal;
an issuing unit configured to issue the activation code for the therapeutic application of the patient in response to the request information; and
a code transmitting unit configured to transmit the activation code issued by the issuing unit to the terminal of the patient.

5. The therapeutic application management system according to claim 1, wherein the limitation unit is configured to limit use of the therapeutic application by transmitting a use limitation code to limit starting of the therapeutic application to the terminal of the patient when the number of days indicated by the use deadline information has elapsed from the date and time at which the therapeutic application has been prescribed based on the date and time information counted by the timekeeping unit.

6. The therapeutic application management system according to claim 1, further comprising an application transmitting unit configured to transmit a therapeutic application in response to a request from a terminal of a patient.

7. The therapeutic application management system according to claim 1, wherein the limitation unit is configured to limit use of the therapeutic application by embedding a user limitation code to disable starting of the therapeutic application in the therapeutic application and transmitting the therapeutic application to the terminal of the patient when the use deadline determined by the use deadline information has elapsed.

8. The therapeutic application management system according to claim 1, further comprising a suggestion unit configured to suggest a use deadline of the therapeutic application prescribed for the patient to the medical worker based on treatment details received from the medical worker using a trained model having learned a relationship between treatment details of the therapeutic application and a use deadline to which the therapeutic application is able to be used.

9. The therapeutic application management system according to claim 8, wherein the storage unit is configured to store the treatment details and a basic number of days until the use deadline to which the therapeutic application is able to be used in association with the treatment details in correlation, and
the suggestion unit is configured to suggest a use deadline obtained by adding a correction number of days estimated using the trained model to the basic number of days corresponding to the treatment details received from the medical worker.

10. The therapeutic application management system according to claim 1, further comprising:
a change unit configured to change a use deadline; and
a change information transmitting unit configured to transmit the changed use deadline and an application ID of the therapeutic application of which the use deadline is changed to the terminal of the patient.

11. The therapeutic application management system according to claim 1, further comprising a change unit configured to change a use deadline,
wherein the limitation unit is configured to limit use of the therapeutic application based on the changed use deadline.

12. The therapeutic application management system according to claim 1, further comprising a notification unit configured to notify the terminal of the patient using the therapeutic application in which the number of days obtained by subtracting a date indicated by the date and time information from a deadline indicated by the use deadline is equal to or less than a predetermined number of days of information indicating that the use deadline of the therapeutic application is imminent.

13. A therapeutic application management method performed by a therapeutic application management system, the therapeutic application management method comprising:
an acquisition step of acquiring an application ID indicating a therapeutic application prescribed for a patient by a medical worker, a patient ID that identifies a patient for whom the therapeutic application is to be prescribed, and use deadline information indicating a deadline to which the therapeutic application is able to be used;
a registration step of registering the application ID that identifies the therapeutic application, the patient ID, and the use deadline information in a storage unit in correlation with each other;
a timekeeping step of counting date and time information; and
a limitation step of limiting use of the therapeutic application transmitted to a terminal of the patient when the date and time information counted in the timekeeping step has passed beyond a date and time determined by the use deadline information.

14. A non-transitory computer readable medium storing therein a management program causing a computer to perform:
an acquisition function of acquiring an application ID indicating a therapeutic application prescribed for a patient by a medical worker, a patient ID that identifies a patient for whom the therapeutic application is to be prescribed, and use deadline information indicating a deadline to which the therapeutic application is able to be used;
a registration function of registering the application ID that identifies the therapeutic application, the patient ID, and the use deadline information in a storage unit in correlation with each other;
a timekeeping function of counting date and time information; and
a limitation function of limiting use of the therapeutic application transmitted to a terminal of the patient when the date and time information counted by the timekeeping function has passed beyond a date and time determined by the use deadline information.
